(19) United States
(12) Patent Application Publication
(10) Pub. No.: US 2007/0198098 A1
(43) Pub. Date: Aug. 23, 2007

(54) Fluid-powered prosthetic apparatus

(76) Inventors: Gerald P. Roston, Saline, MI (US); Renard G. Tubergen, Alto, MI (US)

Correspondence Address: PRICE HENEVELD COOPER DEWITT & LITTON, LLP, 695 KENMOOR, S.E., P O BOX 2567, GRAND RAPIDS, MI, 49501, US

(21) Appl. No.: 11/706,668

(22) Filed: Feb. 15, 2007

Related U.S. Application Data
(60) Provisional application No. 60/774,837, filed on Feb. 17, 2006.

(51) Int. Cl. A61F 2/72; A61F 2/66

(52) U.S. Cl. 623/26; 623/57

(57) ABSTRACT

An improved prosthetic device having a plurality of independently movable members that operate at peak efficiency for a majority of the time that it is in the on state, thereby extending battery life, includes at least two members that are independently movable, a fluid actuator associated with each of the independently movable members for effecting movement, a fluid pump or compressor having a fluid inlet and a compressed or pressurized fluid outlet, an electrical motor coupled to the pump or compressor, a fluid conveying conduit between the pump or compressor outlet and the actuator, a fluid reservoir in communication with the conduit between the pump or compressor outlet and the actuator, and at least one valve associated with each of the independently movable members.

### CROSS REFERENCE TO RELATED APPLICATIONS

This application claims the benefit of provisional patent application Application No. 60/774,837, filed Feb. 17, 2006 by Gerald P. Roston, the entire disclosure of which is incorporated herein by reference.

### BACKGROUND OF THE INVENTION

1. Field of the Invention

This invention relates to prosthetic devices, specifically those with a plurality of motions.

2. Description of the Related Art

In the US there are 90,000 people who, due to birth defect, accident, or disease, have lost the use of one (or both) of their hands. While prosthetic devices exist to assist these individuals, most are little more than glorified pincers which are too heavy and lack functionality. In fact, less than 50% of eligible amputees choose to not wear a prosthetic device because of these limitations. This fact clearly indicates that upper extremity amputees are an underserved group with respect to the technology available to improve their lives.

Most prosthetic hands are large and heavy because they employ a single, permanent magnet electric motor to motivate the hand. Due to the necessity of providing a certain level of force, this implementation practice necessitates the use of a motor that is large (as compared to the available volume), heavy (as compared to a human hand), and expensive. Though the use of a single, large electric motor suffices for current prosthetic device, this practice cannot be extended to hands with multiple, independent motions.

Most prosthetic hands lack functionality because they provide only one motion (degree of freedom). The reasons for this include size/weight constraints (see previous paragraph) and the challenges associated with controlling more than one motion. The control problem has been addressed by Jeffrey Elkins of Elkins Innovations, Inc. in a patent application entitled “Foot-Operated Controller”, Publication No. US-2004-0078091-A1. This published application describes a family of controllers that provide means to control prosthetic devices with multiple degrees of freedom (i.e., independent movements).

Others have endeavored to address the first problem, but that fact that the market is dominated by single motion prosthetic hands indicates a general failure to solve the problem.

U.S. Pat. No. 6,896,704 to Higuchi is focused on specific kinematic finger designs, and does not address the fundamental problem. U.S. Pat. No. 6,676,707 to Yih is similarly focused on kinematic arrangement of prosthetic devices.

U.S. Pat. No. 6,684,754 to Comer discloses an artificial muscle analog that is focused on using inflatable bladders to drive a cable to operate a prosthetic. While this patent teaches the use of a fluidic system for prosthetic control, bladders are inefficient and long-term reliability of the bladder is questionable due to its bearing on a cable. In addition, without the use of an accumulator, this system requires the use of a single, large electric motor.

U.S. Pat. No. 6,558,430 to Nakaya discloses an air-cylinder apparatus for prosthetic limb that is specifically designed to assist with walking. The system described employs a pair of passive cylinder whose mode of operation can be adjusted manually.

U.S. Pat. No. 6,505,870 to Laliberte discloses an actuation system for a highly underactuated gripping mechanism with ten degrees of freedom, which requires only two actuators. One method provided to motivating the mechanism employs fluidic power, however, the notion of energy storage in the system is not disclosed. In addition, the mechanism described is too costly to be commercially viable.

U.S. Pat. No. 5,568,957 to Haugs discloses a device comprised of a plurality of fingers moveable in response to pressurization with a fluid such as hydraulic oil. Unlike the current invention, for which the fluidic operates on the prosthetic indirectly, i.e., motion is created by a fluidic cylinder or motor which is coupled to the prosthetic, this invention employs directly driven deformable members. This approach is power inefficient, the gripping surface is non-rigid, and the volume of oil needed is considerable.

U.S. Pat. No. 5,413,611 to Haslam discloses a computerized electronic hand prosthesis apparatus and method utilizing input, feedback, control, and operating systems configurable to provide precise control and gripping forces corresponding to the particular capabilities and requirements of an individual wearer. This patent describes the current state-of-the-art in hand prosthetics, and as such, as subject to all of the limitation previously discussed.

### SUMMARY OF THE INVENTION

In one aspect of the invention there is provided a prosthetic device having a plurality of independently movable members using a fluid powered system having a single motor (for charging the fluid pressure). The pressurized fluid can be easily transported through or around the device to provide motive force (for example, via the use of a cylinder) where needed. Pressurized fluid energy storage (e.g., using an accumulator) allows the motor to be cycled on and off in such a manner that it operates at peak efficiency a majority of the time that it is in the on state, thereby extending battery life.

In yet another aspect of the invention, there is provided a pneumatically or hydraulically operated prosthetic device having multiple degrees of freedom, in which all pneumatic or hydraulic components are packaged within the confines of the prosthetic device itself to provide the motive force for prosthetic movements. The working fluid can be either gaseous or liquidous.

These and other features, advantages and objects of the present invention will be further understood and appreciated by those skilled in the art by reference to the following specification, claims and appended drawings.

### BRIEF DESCRIPTION OF THE DRAWINGS

FIG. 1 shows a schematic for a hydraulic system that employs a 2-way valve.

FIG. 2 shows a schematic for a pneumatic system that employs a 2-way valve.

FIG. 3 shows a schematic for a hydraulic system that employs a 3-way valve.

FIG. 4 shows a schematic for a pneumatic system that employs a 3-way valve and a low pressure accumulator.

FIG. 5 shows a schematic for a hydraulic system that employs a valve and a manual system recharging capability.

FIG. 6 shows the overall system block diagram.

FIG. 7 shows the state diagram for the embodiments shown in FIGS. 1-4.

FIG. 8 shows the state diagram for the embodiment shown in FIG. 5.

### DETAILED DESCRIPTIONS OF THE PREFERRED EMBODIMENTS

There are two technical issues that have restricted existing prosthetic hands to being limited to a single degree of freedom—the inability to provide a reasonable means for controlling additional degrees of freedom and the engineering challenges inherent in packaging multiple motors, batteries, etc within the volume of the prosthetic device. In the context of this application, the term ‘prosthetic device’ shall mean all components of the artificial limb, including the socket, terminal device, etc.; and the term ‘within’ shall mean inside the outer periphery in such a manner that the device retains its appearance of being natural.

The first problem has been addressed by Jeffrey Elkins of Elkins Innovations, Inc. in a patent application entitled “Foot-Operated Controller,” Publication No. US 2004/0078091 A1. This patent application describes a family of controllers that provide means to control prosthetic devices with multiple degrees of freedom (i.e., independent movement).

The second problem, up to now, has been addressed by employing an electro-mechanical operation to produce the desired gripping and holding functions. While this approach works well for hands with a single degree of freedom, for hands with a plurality of motions, this approach becomes untenable. Problems associated with the current technology include:

1: The use of one motor for each motion. Motors capable of providing the customer-needed level of force are large (in terms of the available space), costly and heavy. Making matters worse is the fact that motors have a very narrow range of operating parameters in which they perform efficiently. Outside this range, much of the energy that goes into the motor is wasted as heat.

2: Difficulty providing linear motion, which is desirable for producing finger motions. With an all-electrical hand, typical options for providing linear motion are a linear motor (which is larger, less efficient, heavier and more costly than the motors described above), a linear solenoid (large, heavy, inefficient, and difficult to control) or a mechanical rotary to linear conversion device (such as a rack and pinion drive which is expensive, large, and heavy).

3: The widely varying demands placed on the battery as a function of the number of motions being simultaneously actuated. Subjecting batteries to such varying loads diminishes the operating time and total life time of the battery.

4: Inability to capture energy: With the all-electrical hand, it is not practical to capture and store non-motor-driven finger motions, thus reducing system efficiency.

As an alternative, fluid powered approaches are considered. There are two manners in which the pressurized fluid can be applied. The first is a direct method, in which the fluid pump is actuated in response to a command for motion. The second is an indirect method, in which the pressurized fluid is stored in an accumulator and is released upon a command for motion.

The direct method offers only minimal advantage as compared to current practice because the battery and pump need to be sized to meet instantaneous demands. The indirect method, however, offers significant advantages because it decouples the demand for power from the use of energy, thereby allowing both the generation and consumption aspects of the system to be individually optimized. Advantages of the indirect, fluid-powered system include:

1: Use of a single electric motor. With an indirect, fluid-powered system, a single electric motor a single fluid pump, and one fluid actuator per motion are required. For the levels of force required, the fluid components are smaller, lighter and cheaper than their electrical counterparts.

2: Use of linear fluidic cylinder. For fluidic systems, linear motions are easy to produce and can be provided by a cylinder, which is compact, light and inexpensive.

3: Known, predictable battery loading. With an indirect, fluid-powered system, the battery/motor combination is designed to operate at maximum efficiency. This is made possible by storing the energy in the accumulator and running the motor only when the accumulator needs to be recharged. This approach also allow the use of a smaller motor since it is not directly driving the finger.

4: Energy recapture: With an indirect, fluid-powered system, using non-driven finger motions to pump fluid back into the accumulators is possible, thereby further increasing the system's efficiency.

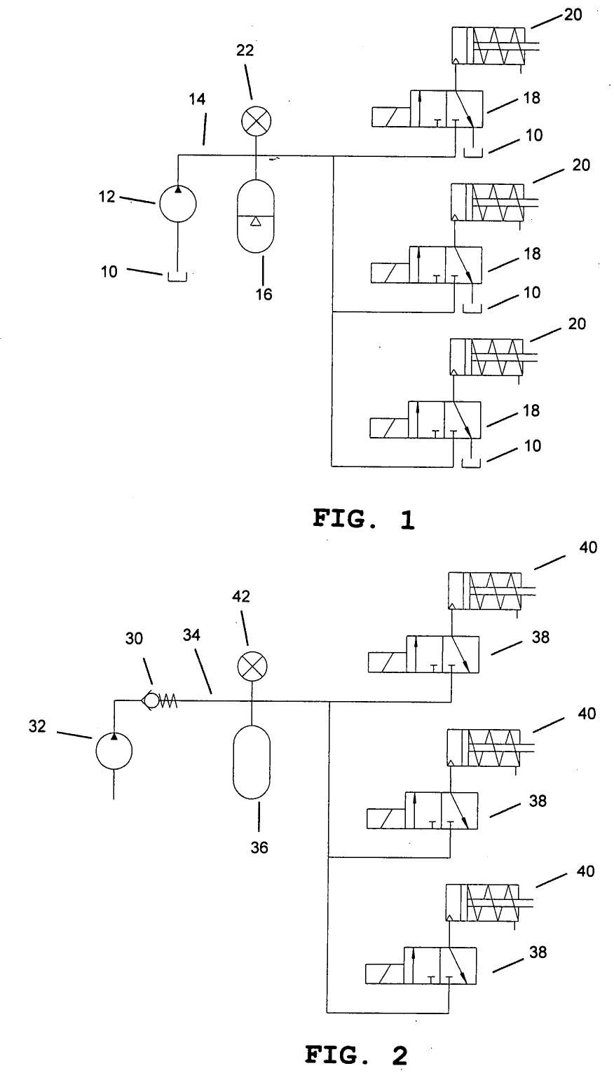

One preferred embodiment of the fluid-powered prosthetic apparatus is shown in FIG. 1. This embodiment employs a liquid, hereafter hydraulic oil, as its working fluid, in a closed-loop system with two-way valves. Oil is drawn from the reservoir 10 into the hydraulic pump 12. When the valves 18 are closed, i.e., not allowing pressurized fluid to flow into the actuators, said pressurized fluid is conveyed from the pump 12 to the accumulator 16 by way of the fluid conveyance 14, whose pressure is monitored by the pressure gauge 22. When one or more valves 18 are open, pressurized fluid flows into the associated actuators 20, causing them to move. The actuators, being connected to the motions of the prosthetic, thereby cause the prosthetic to move. When the valves are closed, the actuators 20 return to their open position and the hydraulic oil contained there within drains to the tank 10.

Another embodiment of the fluid-powered prosthetic apparatus is shown in FIG. 2. This embodiment employs a gas, hereafter air, as its working fluid, in an open-loop system with two-way valves. Air is drawn into the pneumatic pump 32 from atmosphere. When the valves 38 are closed, i.e., not allowing pressurized air to flow into the actuators, said pressurized air is conveyed from the pump 32 to the pressure vessel 36 by way of the fluid conveyance 34, whose pressure is monitored by the pressure gauge 42. The check valve 30 restricts air flow to be directed from the pump 32 into the system. When one or more valves 38 are open, pressurized fluid flows into the associated actuators 40, causing them to move. The actuators, being connected to the motions of the prosthetic, thereby cause the prosthetic to move. When the valves are closed, the actuators 40 return to their open position and the air contained there within is vented to atmosphere.

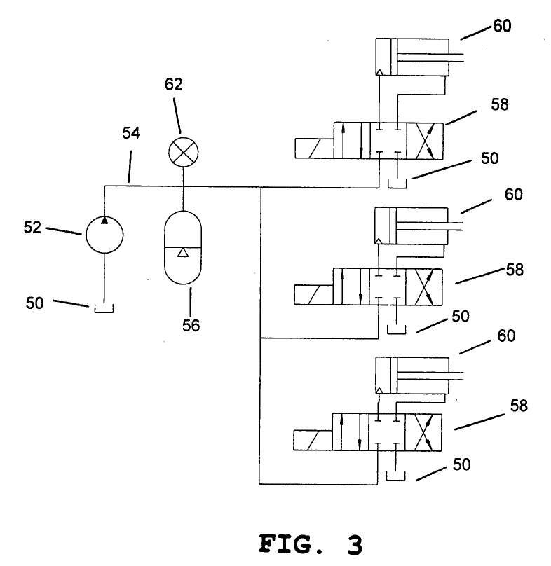

FIG. 3 shows another embodiment of the invention. This embodiment employs a liquid, hereafter hydraulic oil, as its working fluid, in a closed-loop system with three-way valves. Oil is drawn from the reservoir 50 into the hydraulic pump 52. When the valves 58 are closed, i.e., not allowing pressurized fluid to flow into the actuators, said pressurized fluid is conveyed from the pump 52 to the accumulator 56 by way of the fluid conveyance 54, whose pressure is monitored by the pressure gauge 62. When one or more valves 58 are moved to one side, pressurized fluid flows into the associated actuators 60, causing them to move in a direction. The actuators, being connected to the motions of the prosthetic, thereby cause the prosthetic to move. When one or more valves 58 are moved to the other side, the actuators 60 are caused to move in the opposite direction. Oil contained within the actuator is drained to tank 50 when motion occurs.

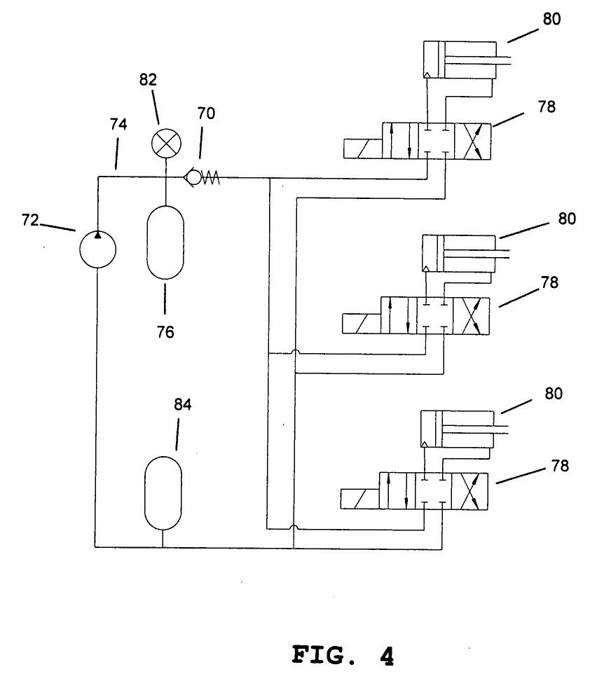

Another embodiment of the fluid-powered prosthetic apparatus is shown in FIG. 4. This embodiment employs a gas, hereafter air, as its working fluid, in an open-loop system with two-way valves. Air is drawn into the pneumatic pump 32 from the low pressure vessel 84. When the valves 78 are closed, i.e., not allowing pressurized air to flow into the actuators, said pressurized air is conveyed from the pump 72 to the high pressure vessel 76 by way of the fluid conveyance 74, whose pressure is monitored by the pressure gauge 82. The check valve 70 restricts air flow to be directed from the pump 72 into the system. When one or more valves 78 are open, pressurized fluid flows into the associated actuators 80, causing them to move. The actuators, being connected to the motions of the prosthetic, thereby cause the prosthetic to move. When the valves are closed, the actuators 80 return to their open position and the air contained there within is stored in the low pressure vessel.

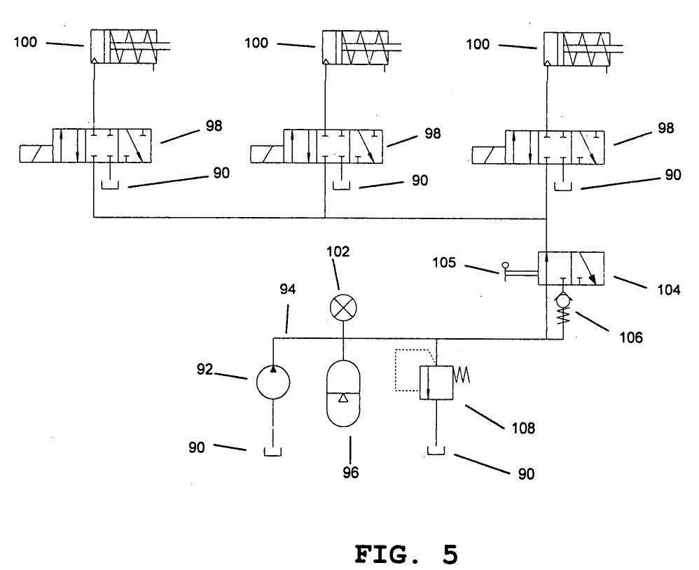

Another embodiment of the fluid-powered prosthetic apparatus is shown in FIG. 5. This embodiment employs a liquid, hereafter hydraulic oil, as its working fluid, in a closed-loop system with two-way valves. Oil is drawn from the reservoir 90 into the hydraulic pump 92. In ‘Not Recharging’ mode, the lever 105 is not activated and the valve 104 is in the left position. When the valves 98 are closed, i.e., not allowing pressurized fluid to flow into the actuators, said pressurized fluid is conveyed from the pump 92 to the accumulator 96 by way of the fluid conveyance 94, whose pressure is monitored by the pressure gauge 102. When one or more valves 18 are open, pressurized fluid flows into the associated actuators 100, causing them to move. The actuators, being connected to the motions of the prosthetic, thereby cause the prosthetic to move. When the valves are closed, the actuators 100 return to their open position and the hydraulic oil contained there within drains to the tank 90. In ‘Recharging’ mode, the lever 105 is activated and the valve 104 is in the right position. With the valve 98 in its left position, fluid is drawn into the actuator 100 be extending the actuator. With the valve 98 in its right position, pressurized fluid is forced back into the system by retracting the actuator 100, thereby forcing oil back into the accumulator 96 by way of the check valve 106. Should too much pressure be created, excess pressure is relieved by the pressure relief valve 108.

There are numerous conformations for prosthetic hands that fall under the purview of this invention. One preferred embodiment is a hand with three independent motions comprising an independently operated thumb, independently operated forefinger and three dependently operated fingers. In this embodiment, all three motions can be provided by fluid-powered actuators. In another embodiment, the hand is comprised of four motions, three as previously described and the fourth being a wrist rotation. In this embodiment, the wrist motion could be provided by a linear or rotary actuator.

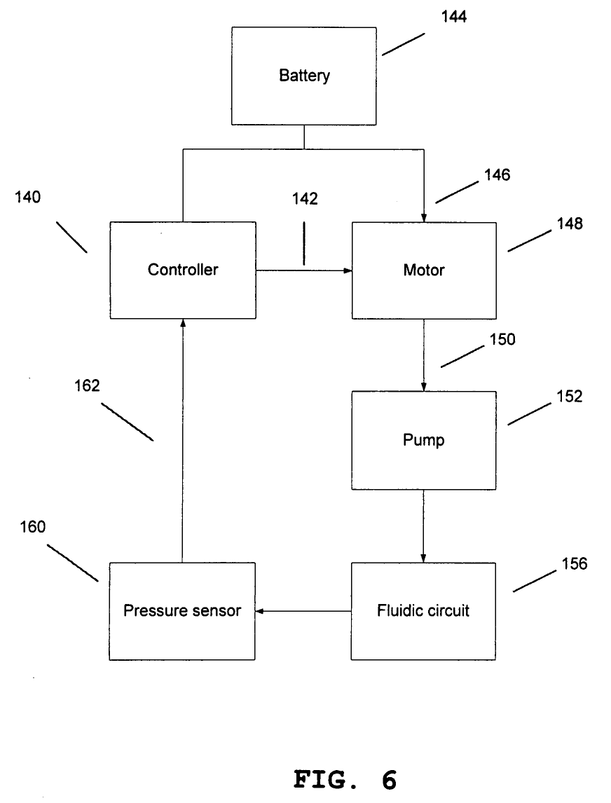

FIG. 6 provides the discloses the overall system functional block diagram. A controller 140, typically a computer-based device, is powered by battery 144 via electrical power connection 146. The controller sends a signal to the motor 148 by way of the electrical connection 142. The motor is also powered by battery 144, which may be located in or on the prosthetic device, via electrical power connection 146. The motor is connected to the pump 152 by a mechanical coupling 150. The pump 152 is a generic representation of the pumps shown in FIGS. 1-5, and numbered 12, 32, 52, 72, and 92. The pump 152 is connected to the fluidic system 156, as shown in FIGS. 1-5. A pressure sensor 160 is incorporated into the fluidic circuit, as shown in FIGS. 1-5 and numbered 22, 42, 62, 82, 102. The pressure sensor 160 provides as its output 162 a signal proportional to the system pressure. This pressure signal is read by the controller 140.

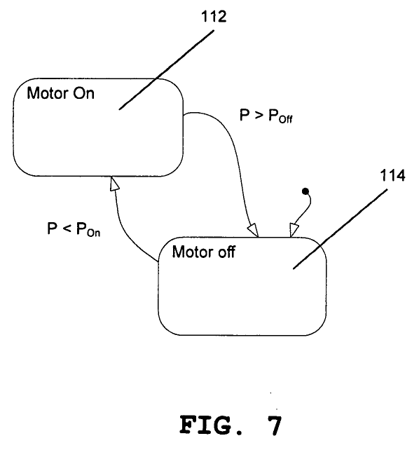

FIG. 7 describes the operation of the embodiments depicted in FIGS. 1-4 and 6. On system start-up, the controller 140 is in Motor Off state 114. If the pressure signal 162 is less than a predetermined value, the system transitions into Motor On state 112. In Motor On state, the electric motor 148 operates, causing the system pressure to increase. When the system pressure exceeds another predetermined value, the system transitions into Motor Off state 114.

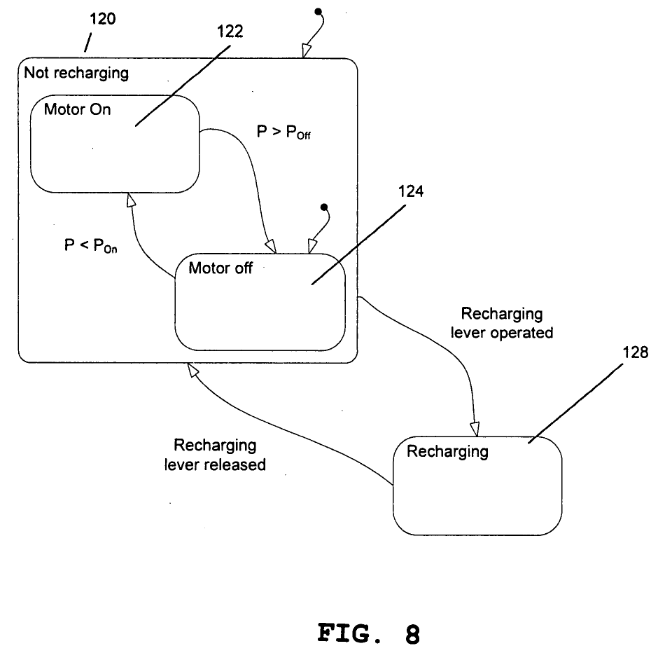

FIG. 8 describes the operation of the embodiments depicted in FIGS. 5 and 6. On system start-up, the controller 140 is in Motor Off state 124, which lies within the Not Manually Pumping super-state 120. If the pressure signal 162 is less than a predetermined value, the system transitions into Motor On state 122. In Motor On state, the electric motor 148 operates, causing the system pressure to increase. When the system pressure exceeds another predetermined value, the system transitions into Motor Off state 124. At any time, if the recharging lever 105 is activated, the motor 148 is turned off (if it is on) and the system transitions into the Accumulator Charging state 128. When the recharging lever 105 is deactivated, the system returns to the Not Manually Pumping super-state 120.

The computer-controlled, fluid powered prosthetic device described herein, addresses all problems associated with exiting devices as described previously. Though the figures presented show three actuators, the system will work with any number of actuators, being only limited by practical design constraints, such as packaging volume and allowable weight.

The embodiment shown in FIG. 1, combined with the block diagram shown in FIG. 6 and the state diagram shown in FIG. 7, provides prosthetic functionality using simple components. The use of the 2-way valve 18 simplifies the design because such valves can be very compact and low cost. With this embodiment, the fingers are either extended or are being contracted.

The embodiment shown in FIG. 2, combined with the block diagram shown in FIG. 6 and the state diagram shown in FIG. 7, provides prosthetic functionality using simple components. The use of the 2-way valve 38 simplifies the design because such valves can be very compact and low cost. With this embodiment, the fingers are either extended or are being contracted. The check valve 30 ensures that the system pressure cannot back-drive the pump. For simplicity, air from the actuators is vented to atmosphere.

The embodiment shown in FIG. 3, combined with the block diagram shown in FIG. 6 and the state diagram shown in FIG. 7, provides prosthetic functionality readily available components. A 3-way valve 58 is used to provide the ability for the actuator 60 to rest in an intermediate position. Another feature of this embodiment is the use of hydraulic pressure, as opposed to a spring, to return the actuator 60 to its fully contracted position.

The embodiment shown in FIG. 4, combined with the block diagram shown in FIG. 6 and the state diagram shown in FIG. 7, provides prosthetic functionality readily available components. A 3-way valve 78 is used to provide the ability for the actuator 80 to rest in an intermediate position. Another feature of this embodiment is the use of pneumatic pressure, as opposed to a spring, to return the actuator 60 to its fully contracted position. In addition, to mitigate the noise associated with the venting of high pressure gas to the atmosphere, this embodiment employs a low pressure vessel to capture the air discharged from the actuator 80. With this embodiment, overall system efficiency is that same as that of the embodiment shown in FIG. 2 because the pressure differential between the high and low pressure is the same.

The embodiment shown in FIG. 5, combined with the block diagram shown in FIG. 6 and the state diagram shown in FIG. 8, provides prosthetic functionality readily available components. This embodiment functions in the same manner as that shown in FIG. 1 when the recharge valve 104 is in the left position. The added feature of this embodiment is the ability to manually operate the actuators thereby forcing high pressure oil back into the accumulator. This ability can greatly extend battery life as it reduces the amount of time the motor needs to operate in order to maintain accumulator charge.

In certain embodiments of the invention, movement of a finger or other movable component of the prosthetic device by external forces (e.g., gravity) could be used to increase fluid pressure in a fluid accumulator located between the pump/compressor and an actuator. The fluid accumulator, as previously stated, acts as an energy reservoir which allows externally derived energy to be stored for later use, thereby possibly further reducing the size and energy requirements for the pump/compressor motor and/or battery. For example, a portion of the energy needed to raise a limb could be recovered by allowing lowering of the limb under the influence of gravity to cause fluid to be pumped back into the accumulator.

The above description is considered that of the preferred embodiments only. Modifications of the invention will occur to those skilled in the art and to those who make or use the invention. Therefore, it is understood that the embodiments shown in the drawings and described above are merely for illustrative purposes and not intended to limit the scope of the invention, which is defined by the following claims as interpreted according to the principles of patent law, including the doctrine of equivalents.

The invention claimed is:

1. A prosthetic device having at least two members that are independently movable with respect to a reference member, a fluid actuator associated with each of the at least two independently movable members for effecting movement of each of the at least two independently movable members with respect to the reference member, a fluid pump or compressor having a fluid inlet and a compressed or pressurized fluid outlet, an electrical motor coupled to the pump or compressor, a fluid conveying conduit between the pump or compressor outlet and the actuator, a fluid reservoir in fluid communication with the conduit between the pump or compressor outlet and the actuator, and at least one valve associated with each of the at least two independently movable members for independently controlling fluid pressure in each of the actuators.

2. The prosthetic device of claim 1, wherein the prosthetic device is configured for attachment to an inoperable or partially amputated human limb.

3. The prosthetic device of claim 1, wherein at least one of the pump or compressor, the motor, or the battery are located within the prosthetic device.

4. The prosthetic device of claim 1 wherein the fluid is a liquid and the reservoir is an accumulator.

5. The prosthetic device of claim 1 wherein the fluid is a gas and the reservoir is a pressure vessel.

6. The prosthetic device of claim 1 wherein fluid can be manually pumped into the fluid reservoir.

7. The prosthetic device of claim 6, wherein the actuator used to recharge the reservoir is also used to actuate one or more members of the prosthetic device.

8. The prosthetic device of claim 6, wherein the actuator used to recharge the reservoir is a separate actuator whose purpose is providing a recharge capability.

9. The prosthetic device of claim 1, wherein the actuator is a linear actuator.

10. The prosthetic device of claim 1, wherein the actuator is a rotary actuator.

11. The prosthetic device of claim 1, further comprising a power source electrically connected to the motor.

12. The prosthetic device of claim 11, wherein the power source is a battery.

13. The prosthetic device of claim 12, wherein the battery is located in or on the prosthetic device.

14. A prosthetic device having a plurality of independently movable members comprising a fluid pump, a pressurized fluid reservoir, an electric motor, and a battery contained within the volume of the prosthetic device, for which the motive force for each of said independently movable members is provided by fluid power which sources from the pressurized fluid reservoir and acts directly on the movable members.

15. The prosthetic device of claim 14, wherein the prosthetic device is an artificial hand configured for a plurality of finger and/or wrist motions.

16. The artificial hand of claim 15 wherein the fluid is a liquid.

17. The artificial hand of claim 15 wherein the fluid is a gas.
